(19) United States
(12) Patent Application Publication
(10) Pub. No.: US 2019/0209142 A1
(43) Pub. Date: Jul. 11, 2019

(54) Sanitary Urine Test Cup Funnel, Especially Suitable for Women

(71) Applicant: Jennifer Louise DeMoss, Plain Dealing, LA (US)

(72) Inventor: Jennifer Louise DeMoss, Plain Dealing, LA (US)

(21) Appl. No.: 16/237,687

(22) Filed: Jan. 1, 2019

Related U.S. Application Data
(60) Provisional application No. 62/613,860, filed on Jan. 5, 2018.

(51) Int. Cl. A61B 10/00

(52) U.S. Cl. CPC A61B 10/007; G01N 33/493

(57) ABSTRACT

A urine test cup funnel has a collection section and an attached outlet section. The collector section has an open top with a curved top edge, preferably having a rounded lip. The collector section has a gradually tapering cross section shape and an open bottom, with the outlet section attached to the open bottom. A urine test cup can be attached to the urine test cup funnel by a clip, preferably integrally molded to the funnel. A woman can hold the urine test cup funnel against her anatomy so as to direct a urine stream into the test cup. The test cup can can be removed and sealed as necessary for testing.

### CROSS REFERENCE TO RELATED APPLICATIONS

This non-provisional United States patent application claims priority to provisional U.S. patent application Ser. No. 62/613,860, filed Jan. 5, 2018, for all purposes. The disclosure of that provisional patent application is incorporated herein in its entirety, to the extent not inconsistent with this disclosure.

### BACKGROUND

### Field of the Invention

This invention relates to apparatus used in connection with obtaining urine samples from persons, typically for medical testing, including but not limited to testing for the presence of drugs and the like in the person's system. As is well known, urine samples are typically collected in a small urine test cup. Urine collection for men is generally no issue. For women, however, placement and holding of the urine test cup in the proper position for sample collection poses issues. The cup may not be properly positioned, with the urine stream missing the cup, and/or with the sample giver getting urine on her hands, etc.

More particularly, the present invention relates to an apparatus comprising a urine test cup funnel, particularly for use by women, to aid in collection of the sample, and providing a safe and sanitary device for women.

Currently, there are a number of urine testing cups of various sizes used by hospitals and agencies. These cups are used to either test immediately and thrown away, or to be sealed and sent to a lab. Also known are urine funnels as one-piece parts of urine collection devices for women to use for camping. Such devices may be multiple use, and relatively messy and unsanitary.

It is desired to have a urine sample collection apparatus designed specifically for women, and that is easy to use, effective and hygienic.

### SUMMARY OF THE INVENTION

The present invention addresses the various problems and limitations of known prior art urine collection systems. A urine test cup funnel, which may be made of plastic, for example blow formed plastic, and/or molded and laminated paper, is provided. Broadly, the urine test cup funnel comprises a collector section and an outlet section. A urine test cup is removably attached to the urine test cup funnel by a clip or similar means. Generally, the urine test cup funnel is used by the woman pressing the funnel against her body in a position to direct the urine flow into the test cup without overspill.

A plastic, particularly clear plastic, embodiment of the urine test cup funnel is especially suited for use in drug testing for employment or law enforcement requirements where visibility for monitoring is important. A paper (which may be biodegradable paper) embodiment is suited for settings in which the employment/law enforcement protocols are not required, but sterile conditions are still desired.

The urine test cup funnel embodying the principles of the present invention addresses various issues posed by the known prior art apparatus, and provides a funnel which is (1) comfortable for use, due to its comfort form design; (2) sanitary, and (3) disposable; and in addition provides a means to use urine test cups without oversplash.

### BRIEF DESCRIPTION OF THE DRAWINGS

FIG. 1 is a view of a urine test cup funnel embodying the principles of the present invention, with a test cup secured in place.

FIG. 2 a view of the urine test cup funnel of FIG. 1, rotated 90 degrees.

FIG. 3 is a more detailed view of the circled area shown in FIG. 2, showing more detail of the clip holding the test cup in place.

### DESCRIPTION OF THE PRESENTLY PREFERRED EMBODIMENT(S)

While various urine test cup funnels can embody the principles of the present invention, with reference to the drawings some of the presently preferred embodiments can be described.

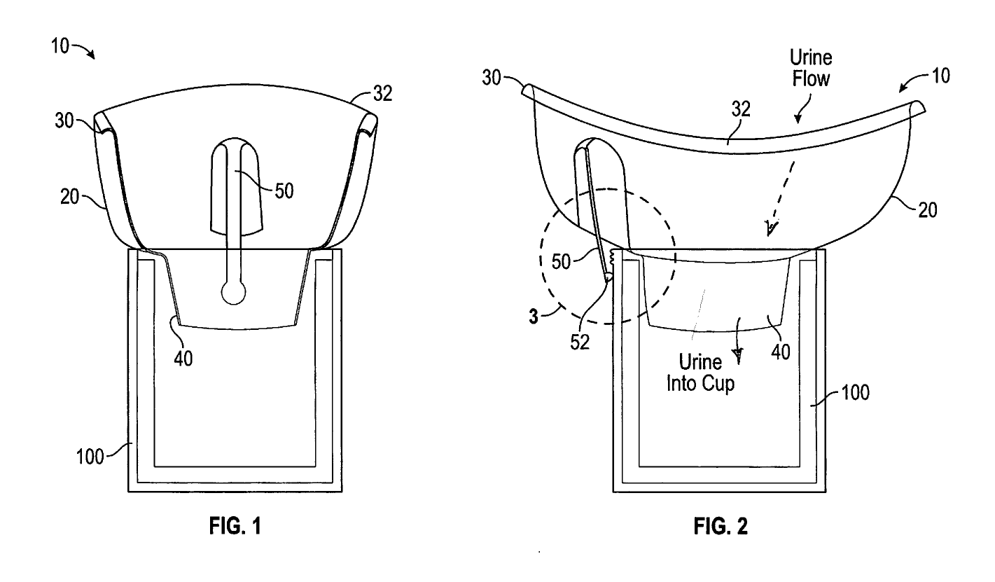

FIG. 1 shows urine test cup funnel 10 in one orientation, with urine test cup 100 attached. Urine test cup funnel 10 comprises a collector section, generally shown as 20, and an outlet section, generally shown as 40. A clip 50, described in more detail below, releasably holds urine test cup 100 in place during use.

Referring to FIGS. 1 and 2, collector section 20 of urine test cup funnel 10 is generally sized and shaped to fit snugly against a woman's anatomy, so as to direct a urine stream into urine test cup 100. Collector section 20 generally comprises an open top and a gradually tapering cross section shape, terminating in an open bottom. Collector section 20 preferably comprises a curved top edge 32, as can be seen in FIG. 2. Preferably, curved top edge 32 comprises a rounded or rolled lip 30 around at least a portion of curved top edge 32 for comfort as it is pressed against the woman's anatomy; in addition, rolled lip 30 provides structural stability for urine cup test funnel 10. It is understood that collector section 20, rolled lip 30 and top edge 32 are sized and shaped as needed to provide a reasonable seal against the woman's anatomy, while still being comfortable. Collector section 20 may be made in different sizes and shapes if needed.

Collector section 20 generally leads to an outlet section 40 attached to the open bottom of collector section 20, as seen in the figures. Outlet section 40 is in its simplest form a short tubular piece, through which urine can flow. Preferably, outlet section 40 is integrally formed with collector section 20, but a multi-piece, attached-together embodiment is possible. Outlet section 40 may be circular or somewhat oval in cross section shape, and is sized and shaped to fit within a standard urine test cup 100, as can be seen in the figures. It can be readily understood that urine flowing into collector section 20 is directed into outlet section 40, and out of outlet section 40 into urine test cup 100, as noted in FIG. 2.

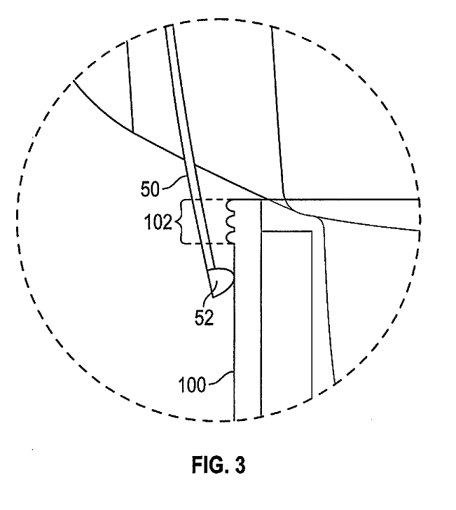

Clip 50 is preferably integrally formed with urine test cup funnel 10, and is formed so as to be biased toward the center part of outlet section 40. As can be seen in the figures, clip 50 is attached at a first end to urine test cup funnel 10, typically to collector section 20. Preferably, clip 50 comprises a raised nose 52 proximal a second end. Referring especially to FIGS. 2 and 3, it can be seen that a urine test cup 100 can be “snapped” into place around outlet section 40, with clip 50 bearing against test cup 100 with sufficient force to hold test cup 100 in place, with a urine sample therein. As can be seen in FIG. 3, certain embodiments of urine test cup 100, typically urine test cups having relatively thinner sidewalls, comprise threads 102 proximal its upper edge (so that a cap may be screwed onto test cup 100 to hold its contents inside), and nose 52 may engage threads 102 or just below threads 102, to better secure urine test cup 100 in place. It is understood that clip 50 is, in effect, spring biased so as to bear against urine test cup 100. This spring biasing is preferably a result of the manufacturing process and material, so as to yield sufficient force to hold urine test cup 100 in place. It is further understood that clip 50 is sufficiently strong to hold urine test cup 100 in place whether a thicker walled urine test cup 100 is used, such as shown in FIG. 2; or a thinner walled urine test cup, for example with ridges or screw threads, as shown in FIG. 3.

While in the preferred embodiment, clip 50 is integrally formed with urine test cup funnel 10, it is understood that same may be separately formed and attached to urine test cup funnel 10 by adhesive or other suitable means. Clip 50 may be formed from the same material (e.g. plastic) as the remainder of urine test cup funnel 10, or may comprise a different material, e.g. steel or the like molded internally or externally to the balance of clip 50.

In another embodiment, a urine collection apparatus, especially suitable for women, is provided. The urine collection apparatus comprises the urine test cup funnel 10 as described above, with a urine test cup 100 attached thereto by clip 50.

### Materials

Urine test cup funnel 10 may be formed from various materials. One material that is currently believed suitable is a plastic, for example a polylactic acid (commonly referred to as “PLA”) bio-plastic, with urine test cup funnel 10 formed by injection molding or similar methods. As is known in the relevant art, PLA bio-plastic is typically a transparent plastic made from corn or dextrose. It is understood that other plastics and methods of forming may be used. Alternatively, another embodiment of urine test cup funnel 10 may be made of paper. Preferably, whether plastic or paper, biodegradable materials are used, for example corn plastic and recycled paper.

### Use of the Urine Test Cup Funnel

Use of urine test cup funnel 10 is straightforward and largely self-explanatory. A urine test cup 100 is secured in place on urine test cup funnel 10 as shown in the drawings, with outlet section 40 positioned within test cup 100. Clip 50 holds urine test cup 100 firmly in place. The user then positions urine test cup funnel 10 against her anatomy so as to capture the urine stream. The apparatus is then removed, and urine test cup 100 can be disengaged from urine test cup funnel 10 by pulling it away from urine test cup funnel 10, overcoming the securing force of clip 50. If applicable, a top can be screwed onto urine test cup 100, and the urine sample therein securely transported for testing without fear of spillage. It can be readily understood that a urine sample can be taken without spillage and in a very sanitary fashion.

### CONCLUSION

While the preceding description contains many specificities, it is to be understood that same are presented only to describe some of the presently preferred embodiments of the invention, and not by way of limitation. Changes can be made to various aspects of the invention, without departing from the scope thereof. For example, size and shape of collector section 20 and outlet section 40 may be varied to suit particular applications; materials may be various types of plastics, papers, or any other suitable material. Multiple clips 50 may be provided for secure fastening of urine test cup 100.

Therefore, the scope of the invention is to be determined not by the illustrative examples set forth above, but by the appended claims and their legal equivalents.

I claim:

1. A urine test cup funnel, comprising:
a collector section comprising an open top and a gradually tapering cross section shape to an open bottom, said collector section further comprising a curved top edge around said open top, said curved top edge comprising a rolled lip around at least a portion of said curved top edge;
an outlet section attached to said open bottom of said collector section, said outlet section sized and shaped to fit within a urine test cup;
a clip comprising first and second ends, a first end attached to said urine test cup funnel, said second end comprising a nose distal from said first end and biased toward said outlet section, said clip sized and positioned so that said nose bears against a urine test cup disposed on said outlet section, whereby said urine test cup is held in place on said urine test cup funnel with sufficient force to support a weight of a urine sample therein.

2. The urine test cup funnel of claim 1, wherein said clip is integrally formed with said urine test cup funnel.

3. The urine test cup funnel of claim 2, wherein said urine test cup funnel is formed from a plastic.

4. The urine test cup funnel of claim 3, wherein said plastic is a polylactic acid bio-plastic, and wherein said urine test cup funnel is formed by an injection molding process.

5. The urine test cup funnel of claim 1, wherein said urine test cup funnel is formed from paper.

6. A urine collection apparatus, especially suitable for women, comprising:
a urine test cup funnel comprising a collector section comprising an open top and a generally tapering cross section area to an open bottom, said collector section further comprising a curved top edge around said open top, said curved top edge comprising a rolled lip around at least a portion of said curved top edge;
an outlet section attached to said open bottom of said collector section, said outlet section sized and shaped to fit within a urine test cup;
a clip comprising first and second ends, a first end attached to said urine test cup funnel, said second end comprising a nose distal from said first end and biased toward said outlet section, said clip sized and positioned so that said nose bears against a urine test cup disposed on said outlet section, whereby said urine test cup is held in place on said urine test cup funnel with sufficient force to support a weight of a urine sample therein; and
a urine test cup disposed around said outlet section and held in place by said clip.

7. The urine collection apparatus of claim 6, wherein said urine test cup comprises threads around an open end thereof, and said nose of said clip bears against said threads.
